(19) United States
(12) Patent Application Publication
(10) Pub. No.: US 2024/0324853 A1
(43) Pub. Date: Oct. 3, 2024

(54) A SUCTION VALVE FOR AN ENDOSCOPE

(71) Applicant: GA Health Medical Devices Limited, Mullingar, Co. Westmeath (IE)

(72) Inventor: Steven Chau, Shek Mun, Shatin (CN)

(21) Appl. No.: 18/292,857

(22) PCT Filed: Jul. 29, 2022
(86) PCT No.: PCT/EP2022/071356

§ 371 Date: Jan. 26, 2024

(30) Foreign Application Priority Data
Jul. 29, 2021 (GB) 2110898.0

(51) Int. Cl. A61B 1/00; A61B 1/015

(52) U.S. Cl. CPC A61B 1/00068; A61B 1/015

(57) ABSTRACT

A suction valve (1) for an endoscope (2) is described. The suction valve comprises a valve housing (5) having a first lumen (8), a lower part (6) configured for mounting in a suction cylinder (4) of an endoscope (2), and an upper part (7), a suction arm (9) extending laterally of the upper part (7) of the valve housing (5) and having a second lumen (10) is fluidic communication with the first lumen (8), and an actuator comprising a valve to open and close the first lumen and an actuator button (18) operatively coupled to the valve, in which the actuator button is collapsible to actuate the valve to open the first lumen. The actuator button (18) is collapsible from an at-rest configuration in which a top surface of the actuator button comprises a first step (23) and a collapsed configuration in which the top surface of the actuator button comprises a first step (23) and a second step (24).

### FIELD OF THE INVENTION

The present invention relates to a suction valve for an endoscope.

### BACKGROUND TO THE INVENTION

Suction valves for endoscopes are designed to be attached to a suction cylinder of a compatible endoscope and function by aspirating fluid from the distal end of the endoscope through the instrument channel. Compatible endoscopes include Olympus bronchoscopes, cytoscopes, rhino-laryngoscopes and pleurascopes models. A suction device for an Olympus bronchoscope is illustrated in FIGS. 3A, 3B and 9 (Olympus MAJ-209). Referring to FIGS. 3A and 3B, the actuation button has a single step design configured to collapse at a mid-point along the length of the button with the result that the shape of the top part of the button remains the same before and after actuation. Referring to FIG. 9, the suction tube has a sharp turn with a radius of curvature that is less than 25% of the length of the suction tube.

US2010/0049001 describes an endoscope sucking apparatus having a main body that connects with a suction pipe forming the suction arm and a suction tube. A button is provided which is capable of being depressed to actuate the apparatus.

U.S. Pat. No. 5,840,015 discloses an endoscope with a controlling apparatus comprising a suction pipe and an insertion pipe. An operation button is provided which is biased upwardly by a compressed coil spring. The button is depressed to open a lumen of the device.

JP23007/014439 describes a suction valve for an ultrasonic endoscope having a first lumen and a pipe forming the sucking arm having a second lumen. The device comprises an operation button.

### SUMMARY OF THE INVENTION

The Applicant has realised that the actuation button of the prior art devices do not provide any tactile feedback to the user and are required to be checked visually during actuation to confirm that the button has been fully depressed and suction actuated. The Applicant has addressed this problem by providing an actuation button for a suction valve having an upper part and a lower part, wherein the upper part is configured to collapse upon actuation by a user from an at rest configuration in which the top surface has one step to a collapsed configuration in which the top surface has two steps that are in close proximity. As the user's finger is in contact with the top surface during actuation, the user will know when the button has been successfully actuated by touch alone due to the feel of the two steps on the top surface upon actuation. This is in contrast with the actuation buttons of the prior art where the lower half of the button is configured to collapse upon actuation with the result that the shape of the top surface is more or less the same before and after actuation, making a tactile determination of actuation difficult if not impossible.

In a first aspect, the invention provides a suction valve for an endoscope, the suction valve of the type comprising:
a valve housing having a first lumen, a lower part configured for mounting in
a suction cylinder of an endoscope, and an upper part,
a suction tube extending laterally of the upper part of the valve housing and having a second lumen is fluidic communication with the first lumen,
an actuator comprising a valve to open and close the first lumen and an actuator button operatively coupled to the valve, in which the actuator button is collapsible to actuate the valve to open the first lumen

Typically, the actuator button is collapsible from an at-rest configuration in which a top surface of the actuator button comprises a first step (or flange) and a collapsed configuration in which the top surface of the actuator button comprises a first and second step.

In any embodiment, the actuator button comprises an annular diaphragm with an upper rim and a through lumen, and the valve comprises a plunger element with a lower part that extends into the first lumen of the valve housing and an upper part that is seated on and covering the upper rim of the annual diaphragm. This presents the first step on the top surface of the actuator button.

In any embodiment, the upper part of the plunger element is wider than the upper rim of the diaphragm. This provides the first step with an overhang.

In any embodiment, the annular diaphragm comprises a top part that is axially collapsible upon actuation and a bottom part that is axially non-collapsible upon actuation.

In any embodiment, the annular diaphragm comprises a middle section that is wider than the top part and bottom part. This middle section presents the second step.

When the button is compressed, the top part of the diaphragm collapses bringing the first and second steps together at the top surface of the collapsed button.

In any embodiment, the suction tube has a first straight section, a proximal straight section, a distal straight section, and an intermediate curved section.

The suction tubes of suction devices of the prior art (See FIG. 9) are cranked intermediate their ends at an angle ø of about 30° and have a distal straight section, a proximal straight section, and an intermediate curved section with a sharp turn (e.g. the radius of curvature is about 20% of the length of the suction tube). The Applicant has realised that the relatively sharp turn on the suction tube of the prior art devices results in unsteady fluid flow with turbulence of separation and back mixing. The Applicant has addressed this problem by providing a suction tube with a longer curved section, generally extending along at least about 30% of the length of the suction arm (for example at least 40%, 50%, 60% or 70%) and in which the radius of curvature of the curved section is at least 50% of the length of the suction tube (i.e. when the suction arm is 8 cm in length the radius of curvature is at least 4 cm, for example 6 or 7 cm).

In any embodiment, the intermediate curved section of the suction tube has a radius of curvature R1 that is at least 60%, 70%, 80%, 90% or 100% of the length of the suction tube L1.

In any embodiment, the suction tube has a length of about 6 to about 10 cm, about 7 to about 9 cm or about 8 cm.

In any embodiment, the radius of curvature of the intermediate curved section is about 5 to about 7 cm, about 5.5 to about 6.5 cm.

In any embodiment, the suction tube is cranked by an angle ø of about 20°-40°. The angle ø is defined between a longitudinal axis of the distal section and the longitudinal axis of the proximal section.

In any embodiment, the intermediate curved section of the suction tube has a radius of curvature R1 that is at least 60% of the length of the suction tube L1.

In any embodiment, the intermediate curved section of the suction tube has a radius of curvature R1 that is at least 70%, 80%, 90% or 100% of the length of the suction tube L1.

In any embodiment, the suction tube has a length of about 6 to about 10 cm, about 7 to about 9 cm or about 8 cm.

In any embodiment, the radius of curvature of the intermediate curved section is about 6 to about 10 cm, about 7 to about 9 cm or about 8 cm.

In any embodiment, the suction tube is cranked by an angle ø of about 20°-40°. The angle ø is defined between a longitudinal axis of the distal section and the longitudinal axis of the proximal section.

The Applicant has also realised that when users rotate the suction tube relative to the endoscope they generally apply torque to the suction arm and not the valve housing. This results in the connection between the suction arm and the valve housing weakening over time, with the connection eventually breaking or cracking. The Applicant has addressed this problem by providing strengthening ribs or webs on each side of a proximal end of the arm, rigidly connecting the proximal end of the arm and the valve housing.

In any embodiment, the suction tube comprises a proximal end joined to the valve housing with a base, top and opposed sides, a distal end, and a second lumen is fluidic communication with the first lumen, and the valve comprises first and second rigid strengthening rib connecting each opposed side of the proximal end of the suction tube with the valve housing. In any embodiment, each rib has a planar triangular shape with a curved end dimensioned to conform to a sidewall of the valve housing and a sidewall dimensioned to conform to a side of the proximal end of the suction tube.

The invention also relates to endoscope comprising a suction valve of the invention. In any embodiment, the endoscope is a bronchoscope.

Other aspects and preferred embodiments of the invention are defined and described in the other claims set out below.

### BRIEF DESCRIPTION OF THE FIGURES

FIG. 1 is a perspective view of a suction valve for a bronchoscope according to the invention attached to a bronchoscope.

FIG. 2 is a perspective view of a suction valve for a bronchoscope according to the invention detached from a bronchoscope.

FIG. 3A (Prior art) is a picture of a suction valve of an Olympus MAJ-209 suction valve showing a user's finger touching the top surface of the actuation button prior to actuation in an at rest configuration.

FIG. 3B (Prior art) is a picture of a suction valve of an Olympus MAJ-209 suction valve showing a user's finger touching the top surface of the actuation button after actuation.

FIG. 4A is a side elevational view of the suction valve of the invention prior to actuation showing the first step on the top surface of the actuation button.

FIG. 4B is a side elevational view of the suction valve of the invention after actuation showing the first and second steps on the top surface of the actuation button.

FIG. 4C is a side elevational view of the suction valve of the invention prior to actuation showing the first step on the top surface of the actuation button.

FIG. 4D is an exploded view of the suction valve of the invention showing the valve housing and suction arm, plunger and diaphragm.

FIG. 5 is a sectional view showing the suction valve of FIG. 4 attached to a bronchoscope prior to actuation.

FIG. 6 is a sectional view showing the suction valve of FIG. 4 attached to a bronchoscope after actuation.

FIG. 7 is a top plan view of the suction valve of FIG. 4.

FIG. 8A is a perspective view of the suction valve of FIG. 4.

FIG. 8B is a detailed view of a distal end of the suction arm and strengthening rib of the suction valve of FIG. 8.

FIG. 9 (Prior art) is a sectional view from above the suction valve of FIG. 3A.

FIG. 10 is a sectional view from above the suction valve of FIG. 4.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

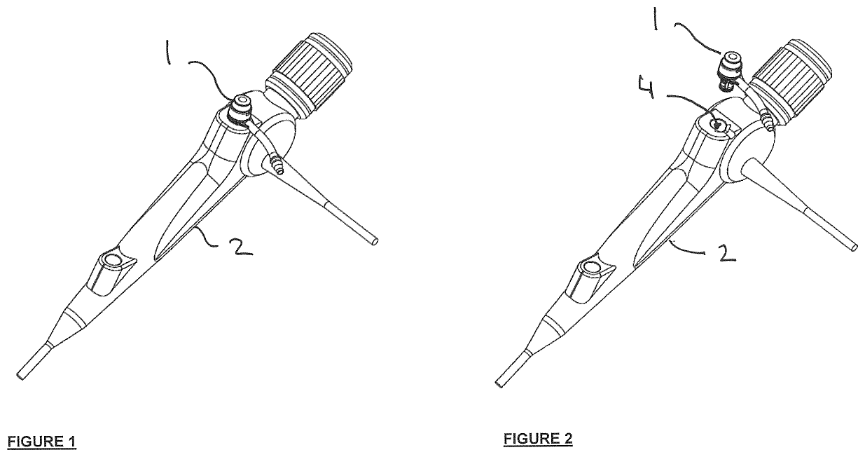

Referring to the drawings, and initially to FIGS. 1 and 2, there is illustrated a suction valve according to the invention indicated generally by the reference numeral 1 and a bronchoscope 2 with a suction cylinder 4. The suction valve is shown coupled to the suction cylinder 4 of the bronchoscope in FIG. 1 and detached from the suction cylinder in FIG. 2.

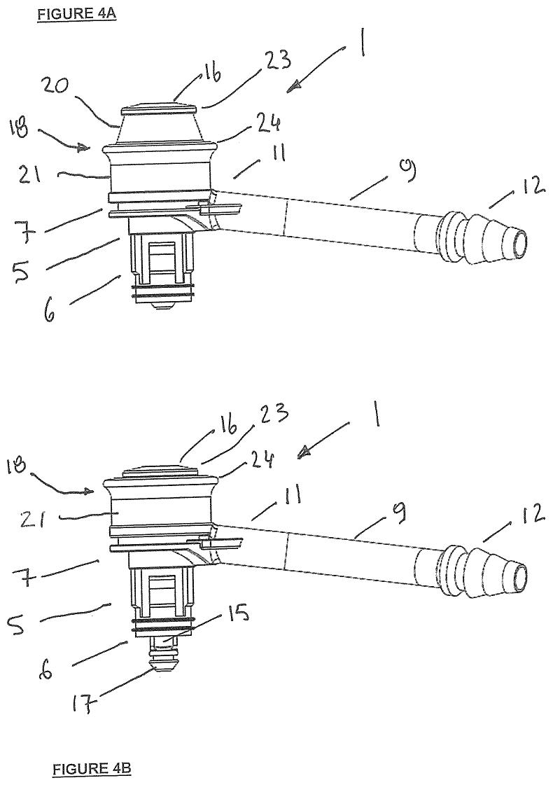

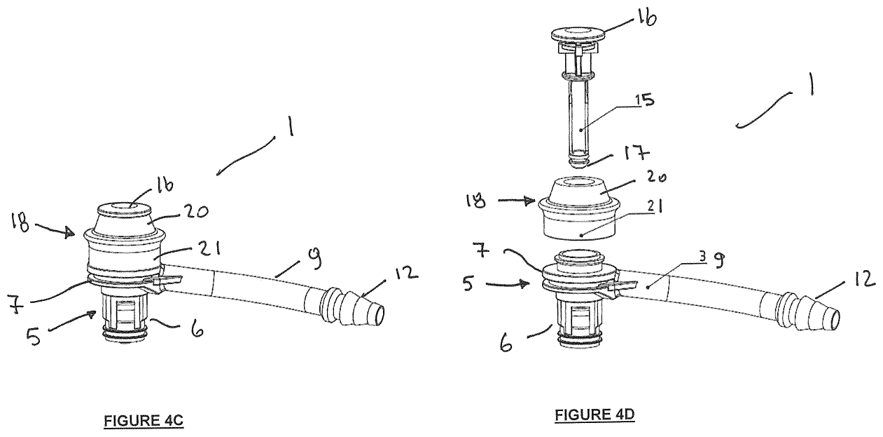

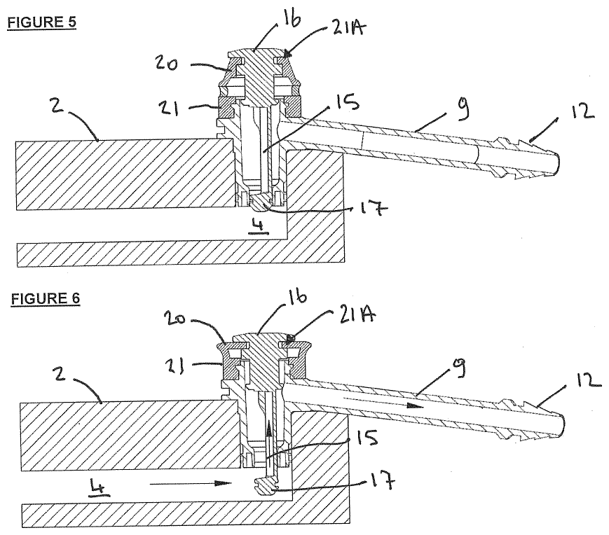

Referring to FIGS. 4 to 6, the suction valve comprises a valve housing 5 with a lower part 6 and upper part 7 and a first lumen 8. A suction arm 9 projects laterally away from the upper part 7 and has a second lumen 10 in fluid communication with the first lumen 8, a distal end 11, and a proximal end 12 configured for coupling with a flexible tube of a mucus specimen trap and vacuum pump (not shown). In FIGS. 5 and 6, the lower part 6 is shown engaged in the suction cylinder 4 of a bronchoscope. A valve to open and close the first lumen is provided and comprises a plunger 15 disposed in the first lumen having an upper head part 16 and a lower lumen closure 17. An actuator button is provided by a compressible rubber diaphragm 18 with a lumen 19 attached to the upper part 7 of the valve housing 5. The diaphragm 18 has an upper section 20 with an annular seat 20A dimensioned to support the distal head part 16 of the plunger 15, and a lower section 21. When a user depresses the actuator button, the diaphragm collapses from the at-rest configuration shown in FIGS. 4A and 5 to the collapsed configuration shown in FIGS. 4B and 6, and the plunger 15 is moved axially downwardly, moving the lumen closure 17 from a position within the first lumen (FIG. 5—closed position) to a position spaced from the first lumen (FIG. 6—open position). This opens the first lumen, allowing fluidic communication between the vacuum pump and the suction cylinder of the bronchoscope via the first and second lumen.

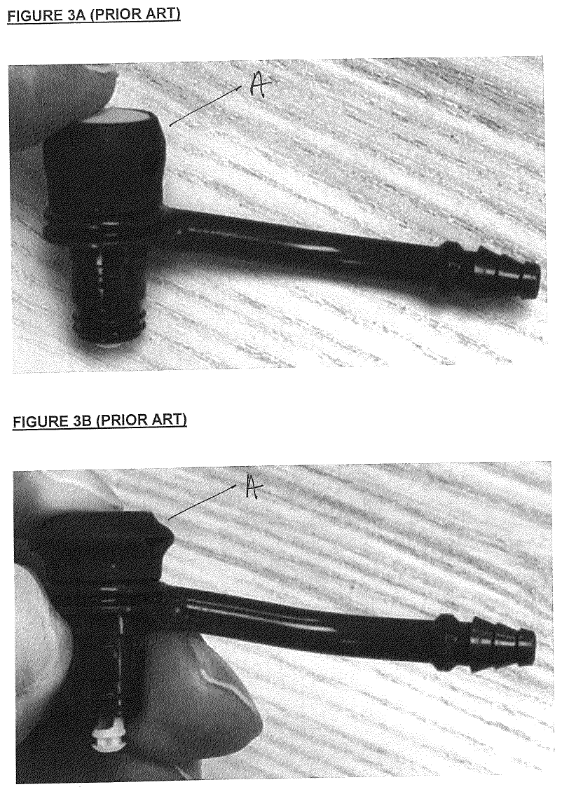

Referring to FIGS. 4A and 4B, the compressible rubber diaphragm 18 is shown in an at-rest configuration in FIG. 4A and a collapsed configuration in FIG. 4B. The upper section 20 of the diaphragm has a first step 23 defined by a periphery of the head 16 of the plunger 15 which extends radially outwardly of the seat 21 of the diaphragm. The lower section 21 of the diaphragm has a second step 24 where the sidewall extends radially inwardly to meet a base of the upper section. The diaphragm is configured such that upon actuation by a user pressing their finger downwardly on a top of the diaphragm, the upper section 20 collapses axially to allow the plunger 16 be depressed and the lower section 21 does not collapse axially, resulting in the first step and second step both being presented on the top surface of the diaphragm. The presence of two steps on the surface allows a user to determine by touch alone when the actuation button has been depressed. Referring to FIGS. 3A and 3B, this is not allowed by the actuation button of the prior art where the lower section of the diaphragm collapses as opposed to the top section, with the result that the shape of the top section A is substantially the same before and after actuation and the user not being able to determine by touch when the button has been actuated. This requires a user to visually confirm that suction has been activated by looking at the button or suction tube, which is an annoyance for a physician. The suction device of the invention obviates this problem by providing an actuation button with tactile feedback.

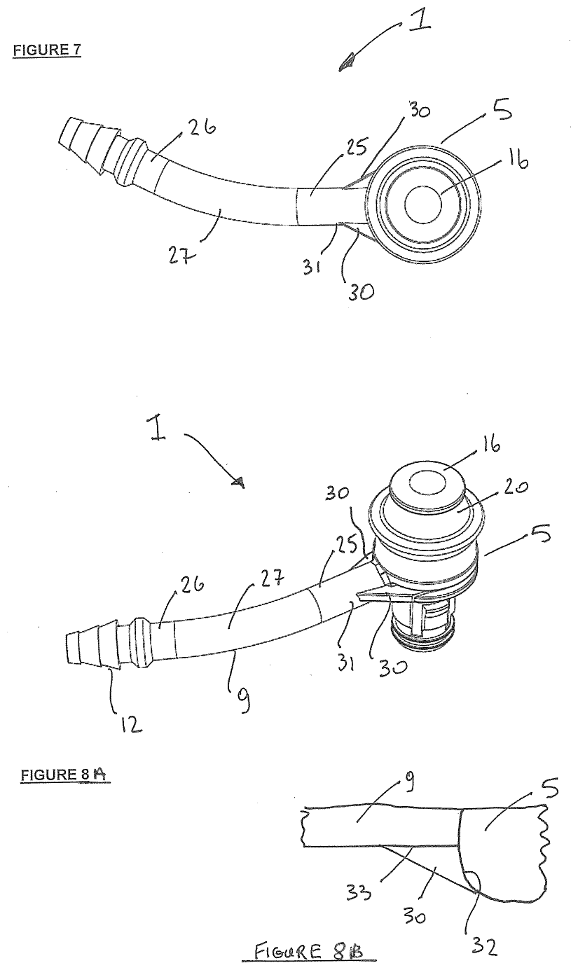

Referring to FIGS. 7, 8A and 8B, the suction arm 9 is illustrated in more detail and comprises a proximal straight section 25 with a proximal end section, a distal straight section 26, and an intermediate curved section 27. Rigid strengthening ribs 30 are provided on each side 31 of the end section 26, strengthening the connection between the suction arm 9 and the valve housing 5. Each rib 30 has a planar generally triangular shape with an end wall 32 that is curved to conform to the valve housing and a sidewall 33 that attaches to each side of the suction arm 9. The ribs are disposed such that a plane of the rib is generally orthogonal to a longitudinal axis of the valve housing.

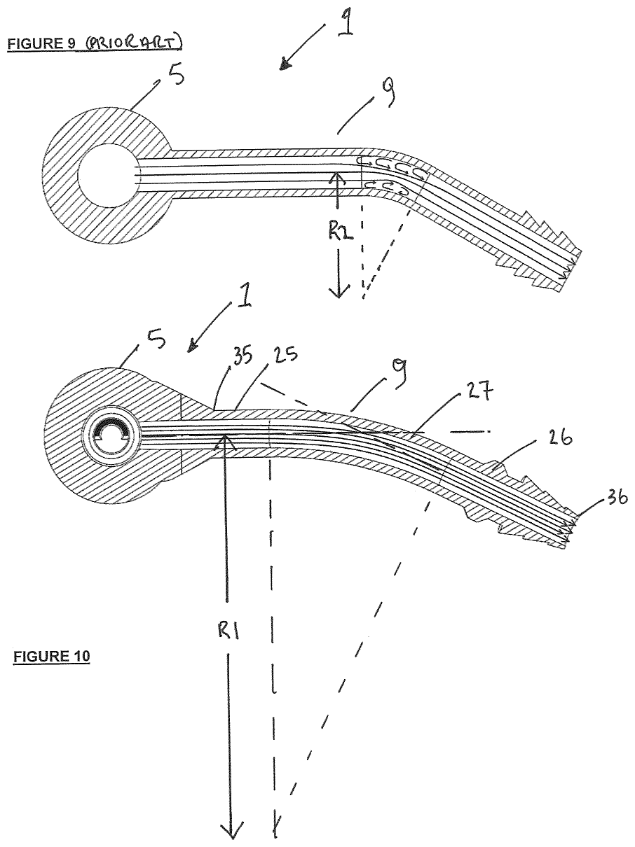

Referring to FIG. 10, the distal straight section 26 of the arm 9 is cranked by about 30° with respect to the proximal straight section 25, and the intermediate curved section 27 has a radius of curvature R1 that is about 100% of the length of the suction tube L1 (where the length L1 is measured from the distal tip 35 of the rib 30 to the distal end 36 of the arm 9). This provides a smooth curved transition from the proximal straight section 25 to the distal straight section 26, allowing steady fluid flow without turbulence. In contrast, the suction arm of the prior art (FIG. 9) has a short intermediate section defining a radius of curvature R2 that is about 25% of the length of the suction tube L2, resulting in unsteady fluid flow with turbulence of separation and back mixing.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A suction valve (1) for an endoscope (2), the suction valve of the type comprising:
a valve housing (5) having a first lumen (8), a lower part (6) configured for mounting in a suction cylinder (4) of an endoscope (2), and an upper part (7);
a suction arm (9) extending laterally of the upper part (7) of the valve housing (5) and having a second lumen (10) is fluidic communication with the first lumen (8); and
an actuator comprising a valve to open and close the first lumen and an actuator button (18) operatively coupled to the valve, in which the actuator button is collapsible to actuate the valve to open the first lumen,
characterised in that the actuator button (18) is collapsible from an at-rest configuration in which a top surface of the actuator button comprises a first step (23) and a collapsed configuration in which the top surface of the actuator button comprises a first step (23) and a second step (24).

2. The suction valve (1) according to claim 1, in which the actuator button comprises an annular diaphragm (18) with a through lumen (19) and an annular seat (21), and the valve comprises a plunger element (15) that extends into the first lumen (8) of the valve housing with a lower lumen closure (17) and an upper distal head (16) that is seated on the annular seat (21) of the annual diaphragm (18) to provide the first step (23).

3. The suction valve (1) according to claim 1, in which the upper distal head (16) of the plunger element is wider than the annular seat (21) of the diaphragm.

4. The suction valve according to claim 1, in which the annular diaphragm (18) comprises an upper section (20) that is axially collapsible upon actuation and a lower section (21) comprising the second step (24) that is axially non-collapsible upon actuation, whereby when the diaphragm is collapsed, the upper section (20) of the diaphragm collapses bringing the first step (23) and second step (24) into proximity at a top surface of the collapsed button.

5. The suction valve (1) according to claim 1, in which the annular diaphragm (18) comprises an upper section (20) that is axially collapsible upon actuation and a lower section (21) comprising the second step (24) that is axially non-collapsible upon actuation, whereby when the diaphragm is collapsed, the upper section (20) of the diaphragm collapses bringing the first step (23) and second step (24) into proximity at a top surface of the collapsed button, in which an upper part of the lower section (21) of the diaphragm (18) projects radially inwardly to meet a lower part of the upper section (20) to provide the second step (24).

6. The suction valve (1) according to claim 1, in which the endoscope is a bronchoscope.

7. The suction valve (1) according to claim 1, in which the suction arm (9) comprises a proximal straight section (25), a distal straight section (26) disposed an at angle ø of 20° to 40° to the proximal straight section, and an intermediate curved section (27) having a radius of curvature R1 that is at least 50% of the length L1 of the suction arm (9).

8. The suction valve (1) according to claim 1, in which the suction arm (9) comprises a proximal straight section (25), a distal straight section (26) disposed an at angle ø of 20° to 40° to the proximal straight section, and an intermediate curved section (27) having a radius of curvature R1 that is at least 50% of the length L1 of the suction arm (9), in which the intermediate curved section (27) has a radius of curvature R1 that is at least 60% of the length L1 of the suction arm (9).

9. The suction valve (1) according to claim 1, in which the suction arm (9) comprises a proximal straight section (25), a distal straight section (26) disposed an at angle ø of 20° to 40° to the proximal straight section, and an intermediate curved section (27) having a radius of curvature R1 that is at least 50% of the length L1 of the suction arm (9), in which the intermediate curved section (27) has a radius of curvature R1 that is 80% to 120% of the length L1 of the suction arm (9).

10. The suction valve (1) according to claim 1, in which the suction tube has a length L1 of about 6 to about 10 cm.

11. The suction valve (1) according to claim 1, in which the radius of curvature R1 of the intermediate curved section is about 6 to about 10 cm.

12. The suction valve (1) according to claim 1, in which the suction arm (9) comprises a proximal straight section (25), a distal straight section (26) disposed an at angle ø of 20° to 40° to the proximal straight section, and an intermediate curved section (27) having a radius of curvature R1 that is at least 50% of the length L1 of the suction arm (9), in which the distal straight section (26) of the suction arm (9) is disposed an at angle ø of 25° to 35° to the proximal straight section (25).

13. The suction valve (1) according to claim 1, in which the suction arm (9) comprises a proximal end section joined to the valve housing (5) and comprising opposed sides (31), wherein first and second rigid strengthening ribs (30) are provided connecting each opposed side of the proximal end of the suction arm with the valve housing.

14. The suction valve (1) according to claim 1, in which the suction arm (9) comprises a proximal end section joined to the valve housing (5) and comprising opposed sides (31), wherein first and second rigid strengthening ribs (30) are provided connecting each opposed side of the proximal end of the suction arm with the valve housing, in which each strengthening rib (30) has a planar shape with a curved end dimensioned to conform to a sidewall of the valve housing and a sidewall dimensioned to conform to a side of the proximal end of the suction tube.

15. The suction valve (1) according to claim 1, in which the suction arm (9) comprises a proximal end section joined to the valve housing (5) and comprising opposed sides (31), wherein first and second rigid strengthening ribs (30) are provided connecting each opposed side of the proximal end of the suction arm with the valve housing, in which the strengthening ribs (30) are disposed such that a plane of each rib is generally orthogonal to a longitudinal axis of the valve housing (5).

16. An endoscope comprising a suction valve (1) of claim 1.

17. A bronchoscope comprising a suction valve (1) of claim 1.
